(19) United States
(12) Patent Application Publication
(10) Pub. No.: US 2025/0134884 A1
(43) Pub. Date: May 1, 2025

(54) METHOD AND PHARMACEUTICAL COMPOSITION FOR TREATING TYPE II DIABETES

(71) Applicants: LUMISTAR BIOTECHNOLOGY, INC., Taipei City (TW); ACADEMIA SINICA, Taipei (TW)

(72) Inventors: Chia-Ning SHEN, Taipei (TW); Edward Po-Fan CHU, Taipei (TW); Jen-Chieh CHENG, Taipei (TW); Yu-Fen CHANG, Taipei City (TW); Wan-Chi SU, Taipei City (TW); Min-Wen CHUNG, Taipei City (TW)

(21) Appl. No.: 18/926,048

(22) Filed: Oct. 24, 2024

Related U.S. Application Data
(60) Provisional application No. 63/593,304, filed on Oct. 26, 2023.

(51) Int. Cl. A61K 31/496; A61P 3/10

(52) U.S. Cl. CPC A61K 31/496; A61P 3/10

(57) ABSTRACT

A method for treating Type II diabetes. The method involves administering to a subject a pharmaceutical composition comprising an effective amount of a 5-HT1A receptor antagonist. The pharmaceutical composition comprises an effective amount of a 5-HT1A receptor antagonist, and pharmaceutically acceptable carriers thereof.

### CROSS-REFERENCE TO RELATED APPLICATION

This application claims the priority benefit of U.S. Provisional Application No. 63/593,304, filed on Oct. 26, 2023. The content of the application is hereby incorporated by reference herein and made a part of this specification.

### BACKGROUND

### 1. Technical Field

The present disclosure relates to a method and a pharmaceutical composition for treating Type II diabetes.

### 2. Description of the Related Art

Diabetes mellitus (DM) is a complex and progressive disease characterized by chronically dysregulated blood glucose levels. The global prevalence of diabetes mellitus is increasing annually. For diabetes mellitus patients, more than 40% will progress to insulin insufficiency due to the failure of pancreatic B cells to produce and secrete insulin at the levels required to regulate glucose metabolism. As a result, substantial global efforts are being directed toward the development of anti-diabetic medications capable of preserving and enhancing the endogenous production of insulin by pancreatic β cells.

Insulin secretion from β cells is triggered by elevated blood glucose levels. Specifically, glucose metabolism leads to an increase in the cytosolic ATP/ADP ratio and the closure of ATP-sensitive K⁺ (KATP) channels. The resulting depolarization of the plasma membrane activates voltage-dependent Ca²⁺ channels, leading to Ca²⁺ influx and the exocytosis of insulin granules. The primary treatment for type 2 diabetes involves administering blood sugar-lowering drugs, such as those that can induce insulin secretion. Current insulin secretagogues, such as derivatives of sulfonylureas and meglitinide, primarily function by binding to the sulfonylurea receptors on the membranes of pancreatic β cells, which results in the closure of ATP-sensitive K⁺ channels (KATP) and subsequently stimulates insulin secretion. In addition to the aforementioned drugs, new pharmaceutical compositions for the treatment of type 2 diabetes are also being explored.

### SUMMARY

In view of the issue above, it is a primary object of the present disclosure to provide a method and a pharmaceutical composition for treating Type II diabetes.

To achieve the above objective, the present disclosure provides a method for treating Type II diabetes. The method involves administering to a subject a pharmaceutical composition comprising an effective amount of a 5-HT1A receptor antagonist.

To achieve the above objective, the present disclosure also provides a pharmaceutical composition for use in the treatment of Type II diabetes. The pharmaceutical composition comprises an effective amount of a 5-HT1A receptor antagonist; and pharmaceutically acceptable carriers thereof.

In at least one embodiment, the 5-HT1A receptor antagonist comprises a WAY-100635 Maleate.

In at least one embodiment, the 5-HT1A receptor antagonist induces insulin secretion by triggering membrane depolarization followed by calcium influx stimulation.

In continuation of the above, according to the method and pharmaceutical composition thereof, which comprises administering an effective amount of 5-HT1A receptor antagonist, which has the effect of promoting secretory insulin, and can be used for the treatment of type 2 diabetes.

These and other objectives of the present disclosure will no doubt become understandable to those of ordinary skill in the art after reading the following detailed description of the preferred embodiment that is illustrated in the various figures and drawings.

### BRIEF DESCRIPTION OF THE DRAWINGS

FIG. 1A is a bar graph depicting the results of insulin secretion measurements by different cell lines in response to glucose challenge;

FIG. 1B is a schematic diagram of the design of the hChR2-EYFP-INS-LUC-K-GECO1 expression construct;

FIG. 2A shows the results of 50 compounds screening using the reporter expression β cells;

FIG. 2B shows the correlation results of repeating FIG. 2A compound screening twice; and

FIG. 3A is the fluorescence intensity graph using the K-GECO1 reporter gene in Min6 cells to monitor glucose stimulation;

FIG. 3B is a representative image using the K-GECO1 reporter gene in Min6 cells after glucose stimulation; and

FIG. 3C is the fluorescence intensity graph using the K-GECO1 reporter gene in Min6 cells to monitor glucose and WAY-100635 Maleate.

FIG. 4 is a relative light unit graph depicting the ability of various 5-HT1A receptor antagonists to induce insulin secretion in MIN6 cells in the presence of a high concentration of glucose.

### DETAILED DESCRIPTION OF THE EMBODIMENTS

The following embodiments are provided to illustrate the present disclosure in detail. A person having ordinary skills in the art can easily understand the advantages and effects of the present disclosure after reading the disclosure of this specification, and also can implement or apply in other different embodiments. Therefore, it is possible to modify and/or alter the following embodiments for carrying out this disclosure without contravening its scope for different aspects and applications, and any element or method within the scope of the present disclosure disclosed herein can combine with any other element or method disclosed in any embodiments of the present disclosure.

In order that the present invention may be more readily understood, certain terms are first defined. In addition, it should be noted that whenever a value or range of values of a parameter are recited, it is intended that values and ranges intermediate to the recited values are also intended to be part of this invention.

As used herein, the singular forms “a,” “an,” and “the” include plural referents, unless expressly and unequivocally limited to one referent. For example, “an element” means one element or more than one element, e.g., a plurality of elements. The term “or” is used interchangeably with the term “and/or” unless the context clearly indicates otherwise.

As used herein, the term “comprising,” “comprises” “include,” “including,” “have,” “having,” “contain,” “containing,” and any other variations thereof are intended to cover a non-exclusive inclusion. For example, when describing an object “comprises” a limitation, unless otherwise specified, it may additionally include other ingredients, elements, components, structures, regions, parts, devices, systems, steps, or connections, etc., and should not exclude other limitations.

As used herein, the term “administering” or “administration” refers to the placement of an active agent into a subject by a method or route which results in at least partial localization of the active agent at a desired site to produce a desired effect. The active agent described herein may be administered by any appropriate route known in the art.

When blood glucose levels rise, the intracellular ATP/ADP ratio increases to metabolize glucose, leading to the closure of ATP-sensitive K⁺ channels (KATP). This causes membrane depolarization, which in turn activates voltage-dependent calcium channels, resulting in calcium influx and the exocytosis of insulin granules. In other words, it triggers pancreatic β cells to secrete insulin in order to regulate blood glucose levels. This disclosure combines optogenetic tools with genetically encoded fluorescent reporters (such as calcium or voltage indicators) along with imaging technology to monitor cellular dynamics. In short, this disclosure utilizes optogenetic technology to observe calcium influx and insulin granule exocytosis in pancreatic β cells, which can be used for screening drugs that promote insulin secretion. For example, this disclosure reveals that the 5-HT1A receptor antagonist and WAY-100635 Maleate, which have not previously been used for diabetes treatment, can promote insulin secretion. The following experimental examples first describe the drug screening platform of this disclosure, followed by further evidence of the effects that specific drugs (5-HT1A receptor antagonist and WAY-100635 Maleate) can achieve.

### Example 1: Generation of a Reporter Construct Expressing β-Cell Line

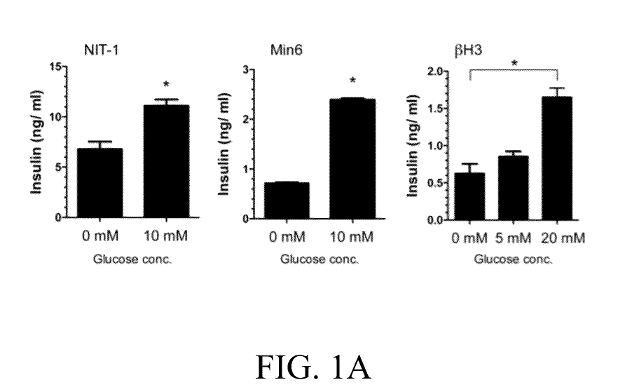

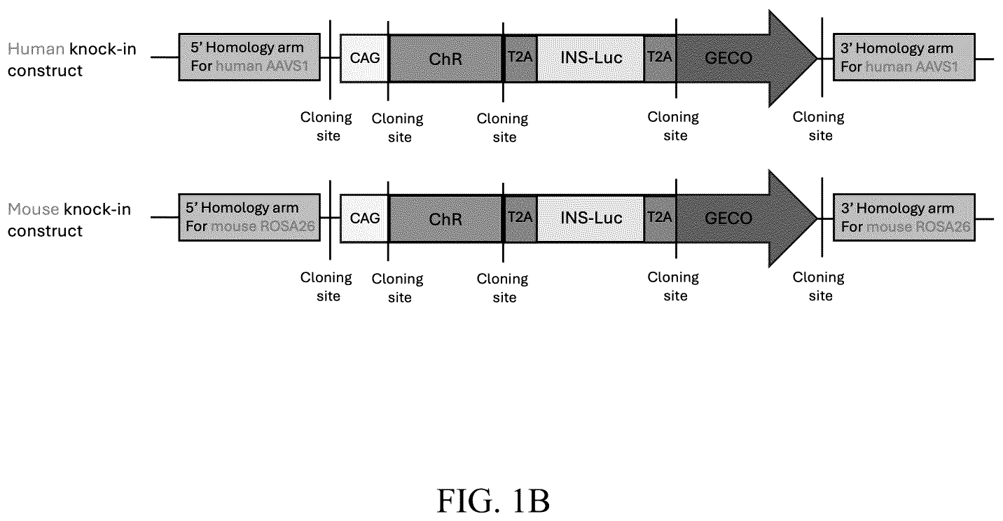

Glucose challenge experiments were first conducted on the mouse β-cell lines (NIT-1 and MIN6) and the human β-cell line (EndoC-βH3). All three cell lines (NIT-1, MIN6 and EndoC-βH3) were capable of secreting insulin in response to varying concentrations of glucose, as shown in FIG. 1A. FIG. 1A is a bar graph depicting the results of insulin secretion measurements by the different cell lines in response to the glucose challenge. The horizontal axis of the bar graph represents glucose concentration, while the vertical axis represents insulin concentration.

To further establish a screening platform, genetically engineered reporters, hChR2-EYFP-hINS-GLuc-K-GECO1, were introduced into the MIN6 cell line. The genetically engineered reporters consist of three components: an INS-luciferase reporter that allows for the detection of secreted insulin via the addition of luciferin substrate. Ca²⁺ indicator that emits fluorescent signal with elevated intracellular Ca²⁺ and a light sensitive Ca²⁺ channel that enables control of Ca²⁺ influx with light.

With the inclusion of a luciferase-tagged insulin construct, hINS-GLuc allows for rapid quantification of secreted insulin in the supernatant for drug screening, unlike the traditional method of measuring insulin via ELISA. This quantification is achieved by measuring the luminescence (relative light units, RLU) upon adding the luciferin of coelenterazine.

Light-sensitive channelrhodopsin-2 (ChR2) were introduced into MIN6 cells together with red fluorescent protein (RFP)-based genetically encoded calcium indicators (GECIs) and INS-luciferase via CRISPR-Cas9 strategy.

To generate hChR2-EYFP-hINS-GLuc-K-GECO1 expressing β cell lines, knock-in constructs (3 μg) and Cas9 expression plasmids (2 μg) were transfected into 7×10⁶ cells using the Amaxa Cell Line Nucleofector Kit T (Lonza, program G-020) with the Amaxa Nucleofector II (Lonza) and plated for 3 days prior to harvest and Fluorescence Activated Cell Sorting (i.e., FACS sorting). Three days post transfection, cells were harvested and resuspended in FACS buffer at 1×10⁷ cells/mL and enhanced yellow fluorescent protein (EYFP) positive cells were acquired on a FACSAriaTMFusion (BD) and plated to expand for an additional 3 days. Further, reporter expressing β cells were expanded and plated in 96 well for 72 hours at 3×10⁴ cells in triplicate.

Two forms of the construct were designed to specifically insert into the “genomic safe harbor” of human and mouse, AAVS1 and ROSA26, respectively, as shown in FIG. 1B. FIG. 1B is a schematic diagram of the design of the hChR2-EYFP-INS-LUC-K-GECO1 expression construct. These two regions are known to be transcriptionally active, allowing for consistent and stable expression of transgenes (Oceguera-Yanez et al., 2016), as well as reducing risk of genomic disruption and insertional mutation. Cutting efficiency of CRISPR-Cas9 gRNAs were determined using mCherry surrogate reporter cell line, resulting in cutting efficiency of 70%-85%, as shown in Table 1.

TABLE 1

| | gRNA1 | gRNA2 |
|---|---|---|
| ROSA26 (mouse) | 85% | 78% |
| AAVS1 (human) | 70%-80% | — |

Table 1 illustrates the results of CRISPR-Cas9 cutting efficiency for ROSA26 and AAVS1 genomic regions.

### Example 2: Compound Screening and Evaluation Using the Reporter Expression B Cells

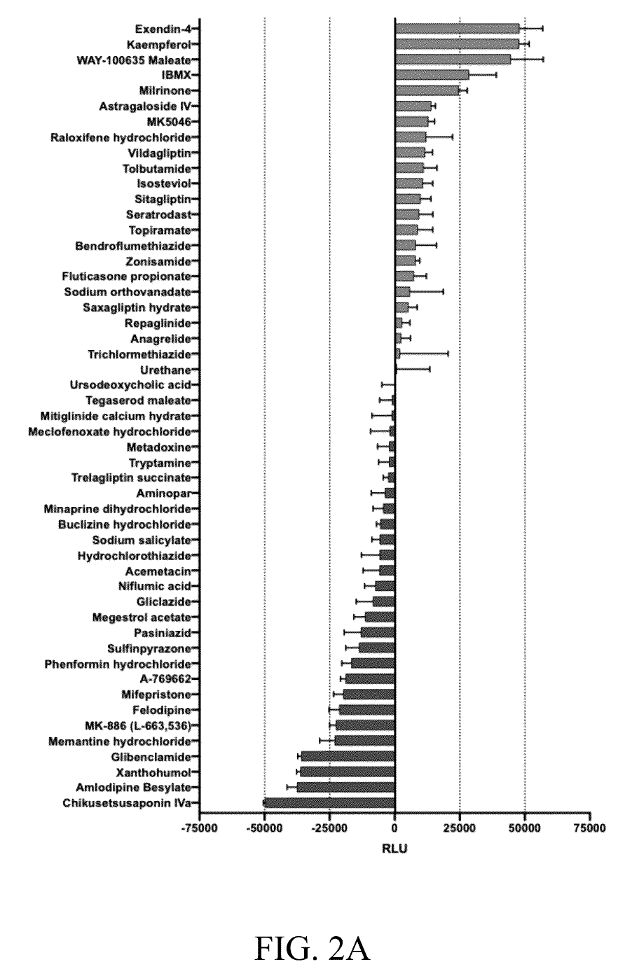

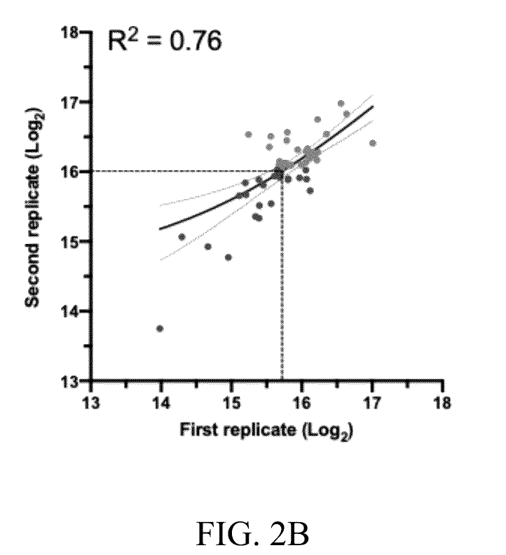

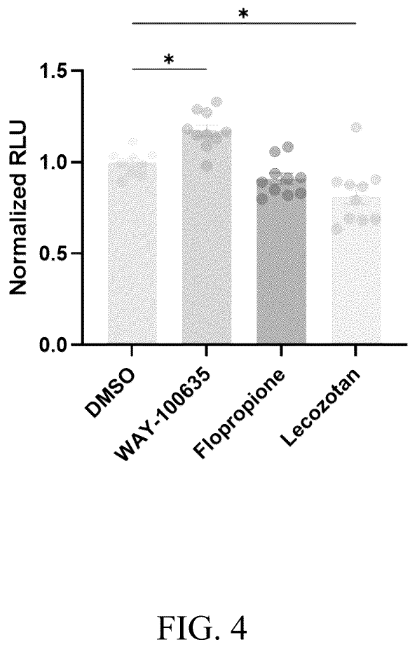

For measuring insulin secretion, the medium will first be removed, and MIN6 cells will then be rinsed in Krebs-Ringer buffer (119 mM NaCl, 4.74 mM KCl, 2.54 mM CaCl₂), 1.19 mM MgSO₄, 1.19 mM KH₂PO₄, 25 mM NaHCO₃, 10 mM Hepes, and 0.1% BSA, pH 7.4) before incubation with or without 20 mM glucose in Krebs-Ringer buffer. Compounds will be added at a final concentration of 25 μM for two hours. At the end of the incubation period, the buffer will be removed, and the cells will then be briefly centrifuged, with the supernatant collected for measuring insulin secretion. Measurement of insulin secretion and C-peptide release will be performed using either an insulin or C-peptide ELISA kit (FIG. 1A), according to the manufacturer's instructions (Mercodia), or by measuring luminescence after adding luciferin (FIGS. 2A, 2B, and 4).

With the establishment of reporter platform in β cell lines, 50 drugs screening with compounds associated with pathways associated with metabolic diseases are conducted. This included inhibitor or agonist against Glucagon-like peptide-1 (GLP-1), Cytochrome c oxidase (COX), Phosphodiesterase (PDE), AMP activated protein kinase (AMPK), dipeptidyl peptidase-4 (DPP-4), calcium and potassium channels, 5-hydroxytryptamine (5-HT) or estrogen/progestogen receptors. As compounds can either “trigger” insulin secretion (i.e., in low glucose condition) or “amplify” insulin secretion (i.e., in high glucose condition). To mimic hyperglycemic condition, the compounds that may amplify insulin secretion under high glucose conditions have been chosen.

Specifically, the compounds comprise Exendin-4, Kaempferol, WAY-100635 maleate, IBMX, Milrinone, Astragaloside IV, MK5046, Raloxifene hydrochloride, Vildagliptin, Tolbutamide, Isosteviol, Sitagliptin, Topiramate, Bendroflumethiazide, Zonisamide, Fluticasone propionate, Sodium orthovanadate, Saxagliptin hydrate, Repaglinide, Anagrelide, Trichlormethiazide, Urethane, Ursodeoxycholic acid, Tegaserod maleate, Mitiglinide calcium hydrate, Meclofenoxate hydrochloride, Metadoxine, Tryptamine, Trelagliptin succinate, Aminopar, Minaprine dihydrochloride, Buclizine hydrochloride, Sodium salicylate, Hydrochlorothiazide, Acemetacin, Niflumic acid, Gliclazide, Megestrol acetate, Pasiniazid, Sulfinpyrazone, Phenformin hydrochloride, A-769662, Mifepristone, Felodipine, MK-886 (L-663,536), Memantine hydrochloride, Glibenclamide, Xanthohumol, Amlodipine Besylate, Chikusetsusaponin IVa, Flopropione, and Lecozotan as shown in FIG. 2A and FIG. 4. FIG. 2A shows the results of 50 compounds screening using the reporter expression β cells; and FIG. 2B shows the correlation results of repeating FIG. 2A compound screening twice. In FIG. 2A, the horizontal axis represents relative light unit (RLU). FIG. 4 shows an evaluation of the ability of various 5-HT1A receptor antagonists to induce insulin secretion.

To enable this screen, we adapted a 96 well format with β cells plated 74 hours prior to compound challenge in triplicates. Post-stimulation, supernatant was aliquoted and transferred into at least 2 empty plates for either storage or luciferase measurement with the addition of coelenterazine. As shown in FIG. 2A, high glucose treatment resulted in elevated insulin secretion (used as baseline “0” for FIG. 2A) and with different compound treatments, both increased (as shown in FIG. 2A, from Exendin-4 to Urethane) and decreased (as shown in FIG. 2A, from Ursodeoxycholic acid to Chikusetsusaponin IVa) insulin secretion was observed when compared to the baseline insulin secretion from high glucose stimulation alone. In addition to technical replicates (triplicates), to ensure reproducibility we have also repeated the screen showing high correlation between the first and second repeat, as shown in FIG. 2B, the correlation of repeats was determined to be R²=0.76.

From this screen, five compounds that significantly enhanced β cell insulin secretion under high glucose condition were identified, as shown in Table 2. Unsurprisingly, Exendin-4, a clinically tested incretin mimetic of the GLP-1 receptor was one of the five compounds. Thus, given Exendin-4 was included as a positive control, this further verified the findings of this screen. Unlike Exendin-4, as a 5-HT1A receptor antagonist, the role of WAY-100635 Maleate in β cells is not well understood. Although the expression of a number of 5-HT receptor have been identified in human islets, receptor-independent pathways have also been recognized in β cells.

TABLE 2

| | Compound | Class |
|---|---|---|
| 1 | Exendin-4 | GLP-1 receptor agonist |
| 2 | Kaempferol | estrogen/progestogen receptor agonist |
| 3 | WAY-100635 maleate | 5-HT receptor agonist |
| 4 | IBMX | PDE inhibitor |
| 5 | Milrinone | PDE inhibitor |

Table 2 illustrates the top five compounds with significant increase in insulin secretion relative to high glucose stimulation.

As shown in FIG. 2A and Table 2, the 5-HT1A receptor antagonist, WAY-100635 Maleate, promotes insulin secretion in a high-glucose condition, which can be applied to the treatment of type 2 diabetes.

### Example 3: Single Cell Ca²⁺ Flux Monitoring Revealed Distinct Ca²⁺ Profile with WAY-100635 Maleate

One component of the reporter construct is a red fluorescent protein (RFP)-based Ca²⁺ indicator (i.e., K-GECO1, reporter with an affinity for calcium ion) that is expressed within the cytoplasm. In this example, an inverted microscope (D1, Zeiss) equipped with a 63 Å˜objective lens (NA 1.4, Zeiss) and a multiwavelength LED light source (pE-4000, CoolLED) will be used. Blue (470 nm) and green (550 nm) excitation will be used to illuminate ChR2-EYFP and K-GECO1, respectively. The green fluorescent protein (GFP) filter set, and the red fluorescent protein (RFP) filter set will be used to confirm the expression of ChR2-EYFP and K-GECO1 in cells. The filter set (Q565lp dichroic mirror, and HQ620/60 emission filter) was used to stimulate ChR2 and observe K-GECO1. Regarding image processing, the raw image data will be extracted using Image J software and processed in Excel (Microsoft) MATLAB (Mathworks) and OriginLab7.5 (Origin).

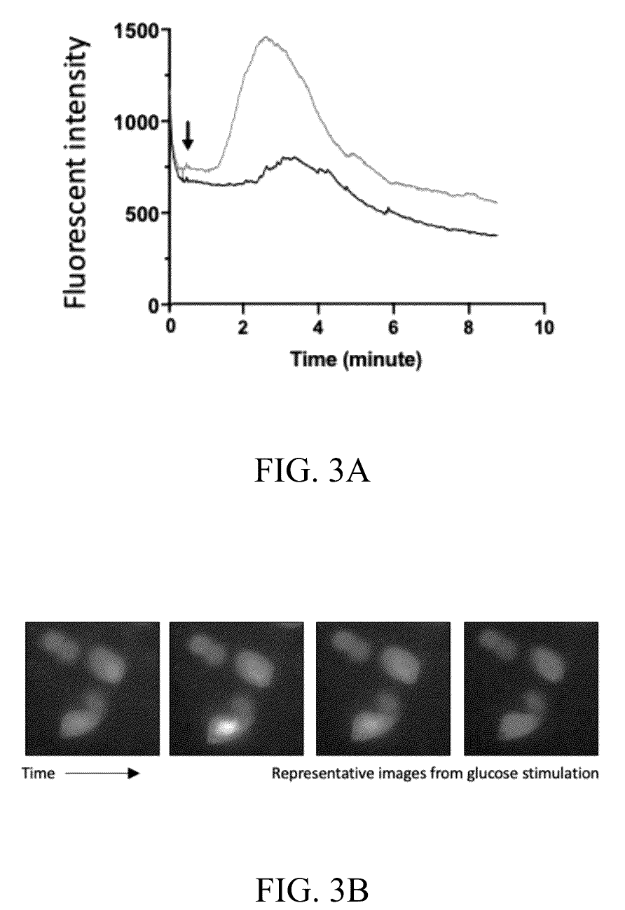

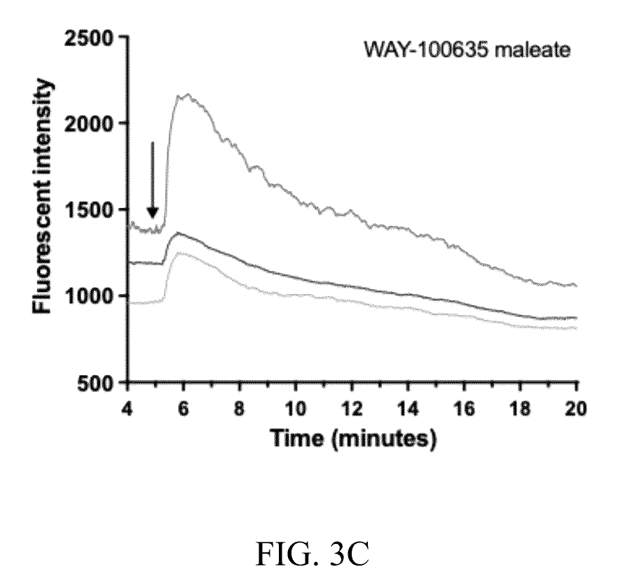

This allowed the detection of Ca²⁺ influx into β cell as a consequence of calcium channel opening. β cells expressing the reporter construct were first stimulated with glucose (e.g., 20 mM glucose) to establish a baseline profile of Ca²⁺ influx over time. Indeed, with glucose stimulation, RFP signal increased over time, peaking at approximately 3 mins post glucose stimulation and gradually returning to baselines at 6 mins, as shown in FIG. 3A. FIG. 3A is the fluorescence intensity graph using the K-GECO1 reporter gene in Min6 cells to monitor glucose stimulation; and FIG. 3B is a representative image using the K-GECO1 reporter gene in Min6 cells after glucose stimulation. In FIG. 3A, the horizontal axis represents time (minutes), the vertical axis represents fluorescence intensity, and an arrow represents the point of stimulation and lines represent individual cells. As it is able to image at single cell resolution, we were able to track individual β cell response to glucose stimulation, showing that individual cells responded differently to glucose treatment as indicated by differences in the magnitude of fluorescent intensity, as shown in the individual lines in FIG. 3A and FIG. 3B.

Further, the integrated Ca²⁺ reporter is used to confirm the ability of WAY-100635 Maleate to induce Ca²⁺ influx in β cells as shown in FIG. 3C, which has not been shown previously. FIG. 3C is the fluorescence intensity graph using the K-GECO1 reporter gene in Min6 cells to monitor glucose and WAY-100635 Maleate. It should be noted that, an arrow represents the point of WAY-100635 stimulation in the presence of glucose and lines represent individual cells. In this example, 20 mM glucose and 25 μM WAY-100635 Maleate are used. Similarly, Ca²⁺ influx is measured as well as insulin secretion when β cells were treated with WAY-100635 Maleate. WAY-100635 Maleate treatment results in significant change in Ca²⁺ influx profile, as shown in FIG. 3C. In other words, the 5-HT1A receptor antagonist, WAY-100635 Maleate, can induce Ca²⁺ influx in β cells to promote insulin secretion.

As described above, β cells can be induced to take up calcium ions in the presence of the 5-HT1A receptor antagonist, WAY-100635 Maleate, which in turn allows for exocytosis of insulin granules, achieving the effect of promoting insulin secretion.

### Example 4: Evaluation of the Effects of Other 5-HT1A Receptor Antagonists on the Stimulation of Insulin Secretion

Other 5-HT1A receptor antagonists were also tested in MIN6 cells to evaluate their ability to induce insulin secretion, as shown in FIG. 4, in the presence of 20 mM glucose. FIG. 4 is a relative light unit graph depicting the ability of various 5-HT1A receptor antagonists to induce insulin secretion in MIN6 cells in the presence of a high concentration of glucose. Among these, only WAY-100635 significantly increased insulin secretion compared to the DMSO control group, while Flopropione and Lecozotan did not show a significant increase, as indicated by the luminescence signals from secreted insulin.

In continuation of the above, according to the method and pharmaceutical composition thereof, which comprises administering an effective amount of 5-HT1A receptor antagonist of WAY-100635 Maleate, which has the effect of promoting secretory insulin, and can be used for the treatment of type 2 diabetes

Although the present disclosure has been explained in relation to its preferred embodiments, it is to be understood that many other possible modifications and variations can be made without departing from the spirit and scope of the disclosure as hereinafter claimed.

What is claimed is:

1. A method for treating Type II diabetes, comprising the following step:
administering to a subject in need thereof a pharmaceutical composition comprising an effective amount of a 5-HT1A receptor antagonist.

2. The method according to claim 1, wherein the 5-HT1A receptor antagonist comprising a WAY-100635 Maleate.

3. The method according to claim 1, wherein the 5-HT1A receptor antagonist induces insulin secretion by triggering membrane depolarization followed by calcium influx stimulation.

4. A pharmaceutical composition for use in the treatment of Type II diabetes, comprising:
an effective amount of a 5-HT1A receptor antagonist; and
pharmaceutically acceptable carriers thereof.

5. The pharmaceutical composition according to claim 4, wherein the 5-HT1A receptor antagonist comprising a WAY-100635 Maleate.

6. The pharmaceutical composition according to claim 4, wherein the 5-HT1A receptor antagonist induces insulin secretion by triggering membrane depolarization followed by calcium influx stimulation.
